(19) United States
(12) Patent Application Publication
(10) Pub. No.: US 2005/0214296 A1
(43) Pub. Date: Sep. 29, 2005

(54) Methods of modulating cytokine activity; related reagents

(75) Inventors: Robert A. Kastelein, Redwood City, CA (US); Terrill K. McClanahan, Sunnyvale, CA (US); Stefan Pflanz, Munich (DE)

Correspondence Address: DNAX RESEARCH, INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 11/058,934

(22) Filed: Feb. 15, 2005

Related U.S. Application Data
(60) Provisional application No. 60/545,762, filed on Feb. 17, 2004.

(51) Int. Cl. A61K 39/395; A61K 48/00; A61K 38/17

(52) U.S. Cl. 424/145.1; 514/12; 514/44

(57) ABSTRACT

Provided are methods of modulating cytokine activity, e.g., for the purpose of treating immune and inflammatory disorders. Also provided are methods of administering agonists or antagonists of IL-27 and IL-27 receptor.

This filing is a U.S. Patent Application which claims benefit of U.S. Provisional Patent Application No. 60/545,762, filed Feb. 17, 2004, which is incorporated herein by reference.

### FIELD OF THE INVENTION

The present invention relates generally to uses of mammalian cytokines. More specifically, the invention discloses a receptor subunit of the EL-27 receptor.

### BACKGROUND OF THE INVENTION

The immune system protects individuals from infective agents, e.g., bacteria, multi-cellular organisms, as well as cancers. This system includes several types of lymphoid and myeloid cells such as monocytes, macrophages, dendritic cells (DCs), eosinophils, T cells, B cells, and neutrophils. These lymphoid and myeloid cells often produce signaling proteins known as cytokines. Immune response includes inflammation, i.e., the accumulation of immune cells systemically or in a particular location of the body. In response to an infective agent or foreign substance, immune cells secrete cytokines which, in turn, modulate immune cell proliferation, development, differentiation, or migration. Immune response sometimes results in pathological consequences, that is, inflammatory disorders. These inflammatory disorders, which involve immune cells and cytokines, include, e.g., psoriasis, rheumatoid arthritis, Crohn's disease, and atherosclerosis (see, e.g., Abbas, et al. (eds.) (2000) Cellular and Molecular Immunology, W. B. Saunders Co., Philadelphia, Pa.; Oppenheim and Feldmann (eds.) (2001) Cytokine Reference, Academic Press, San Diego, Calif.; Kaufmann, et al. (2001) Immunobiol. 204:603-613; Saurez and Schultz-Cheery (2000) Dev. Comp. Immunol. 24:269-283; van Reeth and Nauwynck (2000) Vet. Res. 31:187-213; Garcia-Sastre (2001) Virology 279:375-384; Katze, et al. (2002) Nat. Rev. Immunol. 2:675-687; van Reeth (2000) Vet. Microbiol. 74:109-116; Tripp (2003) Curr. Pharm. Des. 9:51-59).

IL-27 is a heterodimeric cytokine comprising two different subunits, a structure similar to those of IL-12, IL-23, and the CNTF/sCNTFR heterodimer. The two subunits of IL-27 are p28 and Epstein-Barr virus-induced gene 3 (EBI3). IL-27 is expressed by, e.g., antigen presenting cells (APCs), such as monocytes and dendritic cells (DCs). In turn, the expressed IL-27 stimulates proliferation of CD4⁺naïve T cells. Moreover, IL-27 synergizes with IL-12 in provoking CD4^{+naïve T cells to produce interferon-gamma (IFNgamma), a TH}1-type cytokine. IL-27 also upregulates T-bet, a transcription factor specific for TH1-type immune response and, consistent with this, IL-27 downregulates GATA-3, a transcription factor specific for TH2-type immune response. Lipopolysaccharide (LPS) induces expression of both subunits of IL-27 by monocytes and monocyte-derived DCs, indicating a role for IL-27 in innate immunity (Takeda, et al. (2003) J. Immunol. 170:4886-4890; Lucas, et al. (2003) Proc. Natl. Acad. Sci USA. 100:15047-15052; Pflanz, et al. (2002)Immunity 16:779-790; Hashimoto, et al. (2000) Blood 96:2206-2214).

The IL-27 receptor comprises TCCR (also known as WSX-1; WSX-1/TCCR). TCCR/WSX-1 knock out mice (TCCR/WSX-1 KO mice) are distinguished by an impaired TH1-type immune response, e.g., reduced IFNgamma production, increased susceptibility to intracellular pathogens such as Leishmania, Listeria, and Trypanosoma, lower production of TH1-type T cell-dependent antibody (IgG2a subtype) production, abnormal granuloma formation in response to bacillus, lower production of TH1-type T cell-dependent antibody (IgG2a subtype) production. Tuberculosis, sarcoidosis, and Crohn's disease are disorders that involve TH1-type response and granuloma formation. Granuloma formation occurs at the sites of involvement of these diseases. Granulomas from patients with tuberculosis, sarcoidosis, and Crohn's disease express both subunits of IL-27 (see, e.g., Chen, et al. (2000) Nature 407:916-920; Yoshida, et al. (2001) Immunity 15:569-578; Trinchieri, et al. (2003) Immunity 19:641-644; Larousserie, et al. (2004) J. Pathol. 202:164-171; Brombacher, et al. (2003) TRENDS Immunol. 24:207-212).

Subtle variations of immunological pathways involving IL-27 are found, apparently depending on the identity of the pathogen used to challenge the host, and on the time points chosen for study of immune response to infection. For example, some studies of the WSX-1/TCCR knockout mouse demonstrated that the mouse is able to combat infection with an intracellular parasite (Toxoplasma), that the mouse develops excess IFNgamma production, and that IFNgamma production remains upregulated, resulting in lethal inflammation (Chen, et al. (2000) Nature 407:916-920; Villarino, et al. (2003) Immunity 19:645-655; Hamano, et al. (2003) Immunity 19:657-667). According to Trinchieri, et al., supra, IL-27 alone appears not to have a major role in initiating TH1-type response but, instead, stimulates early IFNgamma production without much influencing commitment of T cells to TH1-type differentiation.

There is an unmet need to treat inflammatory and immune disorders, such as psoriasis, arthritis, as well as cancers that resist eradication by the immune system. The present invention fulfils this need by providing methods of using agonists and antagonists of IL-27 or IL-27 receptor.

### SUMMARY OF THE INVENTION

The present invention is based, in part, upon the discovery that an agonist or antagonist of IL-27 or IL-27 receptor modulates response to a number of immune and inflammatory conditions.

The present invention provides a method of modulating an immune disorder or condition, comprising administering an effective amount of an agonist or antagonist of p28, EBI3, or WSX/TCCR, wherein the disorder or condition comprises: a) an inflammatory condition of the skin; b) arthritis; c) Crohn's disease; d) airway hyperreactivity or inflammation; e) atherosclerosis; or f) a cancer or tumor not caused by Epstein-Barr virus. Also provided is the above method, wherein the antagonist inhibits or prevents binding of IL-27 to a receptor comprising a heterodimeric complex of WSX-1/TCCR and gp130.

In another aspect, the invention provides the above method, wherein the inflammatory condition of the skin comprises psoriasis or atopic dermatitis; wherein the arthritis comprises rheumatoid arthritis; osteoarthritis; or psoriatic arthritis; wherein the airway hyperreactivity or inflammation disorder comprises asthma; allergy; or chronic obstructive pulmonary disorder (COPD). Also provided is the above method wherein the cancer or tumor comprises breast cancer; colon cancer; or melanoma; as well as the above method wherein the agonist inhibits or ameliorates the disorder comprising the cancer or tumor; and the above method wherein the cancer or tumor expresses detectably increased amounts, relative to expression by a normal, control tissue, of: a) p28; b) EBI3; or c) or WSX-1/TCCR.

In yet another aspect, the present invention provides the above method wherein the antagonist ameliorates the: a) inflammatory condition of the skin; b) arthritis; c) Crohn's disease; d) airway hyperreactivity or airway inflammation; or e) atherosclerosis.

In another embodiment, the present invention provides a method of modulating an immune disorder or condition, comprising administering an effective amount of an agonist or antagonist of p28, EBI3, or WSX/TCCR, wherein the disorder or condition comprises: a) an inflammatory condition of the skin; b) arthritis; c) Crohn's disease; d) airway hyperreactivity or inflammation; e) atherosclerosis; or f) a cancer or tumor not caused by Epstein-Barr virus; wherein the agonist comprises: IL-27; IL-27 hyperkine; p28; EBI3; or a nucleic acid; or the above method wherein the nucleic acid encodes: IL-27 hyperkine; p28; EBI3; p28 and EBI3; WSX-1/TCCR; or WSX/1/TCCR and gp130; as well as the above method wherein the antagonist comprises a binding composition from an antibody that specifically binds: IL-27; p28; EBI3; WSX-1/TCCR; or a complex of gp130 and WSX-1/TCCR; and the above method wherein the binding composition from an antibody comprises a polyclonal antibody; a monoclonal antibody; a humanized antibody, or a fragment thereof; an Fab, Fv, or F(ab′)₂ fragment; a peptide mimetic of an antibody; or a detectable label. Also provided is the above method wherein the antagonist comprises: a) a soluble receptor derived from WSX-1/TCCR; b) a small molecule; or c) a nucleic acid; as well as the above method wherein the nucleic acid specifically hybridizes with a polynucleotide encoding: p28; EBI3; or WSX-1/TCCR; or the above method wherein the nucleic acid comprises anti-sense nucleic acid or small interference RNA (siRNA).

Yet another aspect of the present invention is the above method wherein administration of the agonist increases and the antagonist decreases expression of: RANKL; TNFalpha; TEASRL; IL-1 alpha or beta; OX40; or APRIL. Also provided is a method of diagnosing the above immune condition or disorder, comprising contacting a binding composition to a biological sample, wherein the binding composition specifically binds to: a) IL-27, p28, EBI3, or WSX-1/TCCR; b) a complex of WSX-1/TCCR and gp130; or c) a nucleic acid encoding p28, EBI3, or WSX-1/TCCR; and measuring or determining the specific binding of the binding composition to the biological sample. Moreover, the present invention also provides a kit for the diagnosis of the immune condition or disorder described above, comprising a compartment and a binding composition that specifically binds to: a) IL-27, p28, EBI3, or WSX-1/TCCR; b) a complex of WSX-1/TCCR and gp130; or c) a nucleic acid encoding p28, EBI3, or WSX-1/TCCR;

### DETAILED DESCRIPTION OF THE PREFERRED EMBODIMENTS

As used herein, including the appended claims, the singular forms of words such as “a,” “an,” and “the,” include their corresponding plural references unless the context clearly dictates otherwise.

All references cited herein are incorporated herein by reference to the same extent as if each individual publication or patent application was specifically and individually indicated to be incorporated by reference.

I. Definitions.

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compositions derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Proliferative activity” encompasses an activity that promotes, that is necessary for, or that is specifically associated with, e.g., normal cell division, as well as cancer, tumors, dysplasia, cell transformation, metastasis, and angiogenesis.

“Administration” and “treatment,” as it applies to an animal, human, experimental subject, cell, tissue, organ, or biological fluid, refers to contact of an exogenous pharmaceutical, therapeutic, diagnostic agent, compound, or composition to the animal, human, subject, cell, tissue, organ, or biological fluid. “Administration” and “treatment” can refer, e.g., to therapeutic, placebo, pharmacokinetic, diagnostic, research, and experimental methods. “Treatment of a cell” encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an IL-27 agonist or IL-27 antagonist to a human or animal subject, a cell, tissue, physiological compartment, or physiological fluid. “Treatment of a cell” also encompasses situations where the IL-27 agonist or IL-27 antagonist contacts IL-27 receptor (heterodimer of WSX-1/TCCR and gp130), e.g., in the fluid phase or colloidal phase, as well as situations where the agonist or antagonist contacts a fluid, e.g., where the fluid is in contact with a cell or receptor, but where it has not been demonstrated that the agonist or antagonist contacts the cell or receptor.

“Binding composition” refers to a molecule, small molecule, macromolecule, antibody, a fragment or analogue thereof, or soluble receptor, capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to a target. “Binding composition” may also refer to a molecule in combination with a stabilizer, excipient, salt, buffer, solvent, or additive, capable of binding to a target. “Binding” may be defined as an association of the binding composition with a target where the association results in reduction in the normal Brownian motion of the binding composition, in cases where the binding composition can be dissolved or suspended in solution.

“Conservatively modified variants” applies to both amino acid and nucleic acid sequences. With respect to particular nucleic acid sequences, conservatively modified variants refers to those nucleic acids which encode identical or essentially identical amino acid sequences or, where the nucleic acid does not encode an amino acid sequence, to essentially identical nucleic acid sequences. Because of the degeneracy of the genetic code, a large number of functionally identical nucleic acids may encode any given protein.

As to amino acid sequences, one of skill will recognize that an individual substitution to a nucleic acid, peptide, polypeptide, or protein sequence which substitutes an amino acid or a small percentage of amino acids in the encoded sequence for a conserved amino acid is a “conservatively modified variant.” Conservative substitution tables providing functionally similar amino acids are well known in the art. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157: 105-132):
(1) Hydrophobic: Norleucine, Ile, Val, Leu, Phe, Cys, or Met;
(2) Neutral hydrophilic: Cys, Ser, Thr;
(3) Acidic: Asp, Glu;
(4) Basic: Asn, Gln, His, Lys, Arg;
(5) Residues that influence chain orientation: Gly, Pro;
(6) Aromatic: Trp, Tyr, Phe;
(7) Small amino acids: Gly, Ala, Ser.

“Derived” can be used to describe, e.g., deriving the structure of a peptide, oligopeptide, or polypeptide from a parent peptide, oligopeptide, or polypeptide, such as an antibody. In this context, derived encompasses, e.g., peptide structures where the peptide has the same sequence as a sequence found within the parent, e.g., where the peptide is identical to the parent but with a truncation at the N-terminus, C-terminus, or both N- and C-termini of the parent, or with a truncation and a fusion, or with a fusion only. Derived also means that the peptide has the same sequence as found in the parent, but with conservative amino acid changes, or with deletions or insertions, where the deletions or insertions preserve a biological property in the peptide that is inherent in the parent. “Derived” encompasses situations where the peptide or polypeptide is synthesized using the parent as a starting compound, and where the peptide or polypeptide is synthesized de novo, using the structure of the parent as a guide.

“Effective amount” or “therapeutically effective amount” means an amount sufficient to ameliorate a symptom or sign of a disorder or physiological condition or an amount sufficient to permit or facilitate a diagnosis of the disorder or physiological condition. An effective amount for a particular patient or veterinary subject may vary depending on factors such as the condition being treated, the overall health of the patient, the method route and dose of administration and the severity of side affects (see, e.g., U.S. Pat. No. 5,888,530 issued to Netti, et al.). An effective amount can be the maximal dose or dosing protocol that avoids significant side effects or toxic effects. The effect will result in an improvement of a diagnostic measure, parameter, or detectable signal by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

“Exogenous” refers to substances that are produced outside an organism, cell, or human body, depending on the context. “Endogenous” refers to substances that are produced within a cell, organism, or human body, depending on the context.

“Disorder” refers to a pathological state, or a condition that is correlated with or predisposes to a pathological state. “Infectious disorder” refers, e.g., to a disorder resulting from a microbe, bacterium, parasite, virus, and the like, as well as to an inappropriate, ineffective, or pathological immune response to the disorder. “Oncogenic disorder” encompasses a cancer, transformed cell, tumor, displasia, angiogenesis, metastasis, and the like, as well as to an inappropriate, ineffective, or pathological immune response to the disorder.

“Effective amount” means, e.g., an amount of an IL-27 agonist, IL-27 antagonist, binding compound or binding composition, sufficient to ameliorate a symptom or sign of a disorder, condition, or pathological state. “Effective amount” also relates to an amount of an IL-27 agonist, antagonist, or binding compound or composition, sufficient to allow or facilitate the diagnosis of a symptom or sign of a disorder, condition, or pathological state.

“Inhibitors” and “antagonists” or “activators” and “agonists” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a composition that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

“Expression” refers to a measure of mRNA or polypeptide encoded by a specific gene. Units of expression may be a measure of, e.g., the number of molecules of MRNA or polypeptide/mg protein, the number of molecules of mRNA or polypeptide/cell, in measurements of expression by cell, tissue, cell extract, or tissue extract. The units of expression may be relative, e.g., a comparison of signal from control and experimental mammals or a comparison of signals with a reagent that is specific for the MRNA or polypeptide versus with a reagent that is non-specific.

“Hybridization” that is specific or selective typically occurs when there is at least about 55% homology over a stretch of at least about 30 nucleotides, preferably at least about 75% over a stretch of about 25 nucleotides, and most preferably at least about 90% over about 20 nucleotides (see, e.g., Kanehisa (1984) Nucleic Acids Res. 12:203-213). Hybridization under stringent conditions, e.g., of a first nucleic acid to a second nucleic acid, are those that: (1) Employ low ionic strength and high temperature for washing, for example, 0.015 M sodium chloride/0.0015 M sodium citrate/0.1% sodium dodecyl sulfate at 50° C.; (2) Employ during hybridization a denaturing agent, such as formamide, for example, 50% (vol/vol) formamide with 0.1% bovine serum albumin/0.1% Ficoll® (Sigma-Aldrich, St. Louis, Mo.)/0.1% polyvinylpyrrolidone/50 mM sodium phosphate buffer at pH 6.5 with 750 mM sodium chloride, 75 mM sodium citrate at 42° C.; (3) Employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 ng/ml), 0.1% SDS, and 10% dextran sulfate at 42° C., with washes at 42° C. in 0.2×SSC and 0.1% SDS; or (4) Employ a buffer of 10% dextran sulfate, 2×SSC (sodium chloride/sodium citrate), and 50% formamide at 55° C., followed by a high-stringency wash consisting of 0.1×SSC containing EDTA at 55° C. (U.S. Pat. No. 6,387,657 issued to Botstein, et al.).

Stringent conditions for hybridization of nucleic acids are a function of salt, temperature, organic solvents, and chaotropic agents. Stringent temperature conditions will usually include temperatures in excess of about 30° C., more usually in excess of about 37° C., typically in excess of about 45° C., more typically in excess of about 50° C., preferably in excess of about 65° C., and more preferably in excess of about 70° C. Stringent salt conditions will ordinarily be less than about 1 M, more ordinarily less than about 500 mM, usually less than about 400 mM, more usually less than about 300 mM, typically less than about 200 mM, preferably less than about 100 mM, and more preferably less than about 80 mM, even down to less than about 20 mM. However, the combination of parameters is more important than the measure of any single parameter (Wetmur and Davidson (1968) J. Mol. Biol. 31:349-370).

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist irradication by the immune system. “Cancerous condition” includes, e.g., cancer, cancer cells, tumors, angiogenesis, and precancerous conditions such as dysplasia.

“Inflammatory disorder” means a disorder or pathological condition where the pathology results, in whole or in part, from, e.g., a change in number, change in rate of migration, or change in activation, of cells of the immune system. Cells of the immune system include, e.g., T cells, B cells, monocytes or macrophages, antigen presenting cells (APCs), dendritic cells, microglia, NK cells, NKT cells, neutrophils, eosinophils, mast cells, or any other cell specifically associated with the immunology, for example, cytokine-producing endothelial or epithelial cells.

“Inflammatory disorder” means a disorder or pathological condition where the pathology results, in whole or in part, from an increase in the number and/or increase in activation of cells of the immune system, e.g., of T cells, B cells, monocytes or macrophages, alveolar macrophages, dendritic cells, NK cells, NKT cells, neutrophils, eosinophils, or mast cells.

“Knockout” (KO) refers to the partial or complete reduction of expression of at least a portion of a polypeptide encoded by a gene, e.g., the p28 or EBI3 subunit of IL-27, where the gene is endogenous to a single cell, selected cells, or all of the cells of a mammal. KO also encompasses embodiments where biological function is reduced, but where expression is not necessarily reduced, e.g., a p28KO polypeptide comprising an expressed p28 polypeptide that contains an inserted inactivating peptide, oligopeptide, or polypeptide. Disruptions in a coding sequence or a regulatory sequence are encompassed by the knockout technique. The cell or mammal may be a “heterozygous knockout”, where one allele of the endogenous gene has been disrupted. Alternatively, the cell or mammal may be a “homozygous knockout” where both alleles of the endogenous gene have been disrupted. “Homozygous knockout” is not intended to limit the disruption of both alleles to identical techniques or to identical outcomes at the genome. Included within the scope of this invention is a mammal in which one or both p28 alleles have been knocked out.

“Ligand” refers, e.g., to a small molecule, peptide, polypeptide, and membrane associated or membrane-bound molecule, or complex thereof, that can act as an agonist or antagonist of a receptor. “Ligand” also encompasses an agent that is not an agonist or antagonist, but that can bind to the receptor without significantly influencing its biological properties, e.g., signaling or adhesion. Moreover, “ligand” includes a membrane-bound ligand that has been changed, e.g., by chemical or recombinant methods, to a soluble version of the membrane-bound ligand. By convention, where a ligand is membrane-bound on a first cell, the receptor usually occurs on a second cell. The second cell may have the same or a different identity as the first cell. A ligand or receptor may be entirely intracellular, that is, it may reside in the cytosol, nucleus, or some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

A “first polypeptide chain” and a “second polypeptide chain” refers to two polypeptide chains not linked together by way of a classical peptide bond. Typically, the first polypeptide chain comprises an N-terminus and C-terminus, and the second polypeptide chain comprises another N-terminus and another C-terminus, that is, altogether there are two N-termini and two C-termini. The first polypeptide chain can be encoded by a first vector, while the second polypeptide chain can be encoded by a second vector. The first polypeptide chain and second polypeptide chain can be encoded by one vector, where a first promoter can be operably linked with the first polypeptide chain and a second promoter can be operably linked with the second polypeptide chain or, in another embodiment, expression of both the first and second polypeptide chains can be operably linked to the same promoter.

“Sensitivity,” e.g., sensitivity of receptor to a ligand, means that binding of a ligand to the receptor results in a detectable change in the receptor, or in events or molecules specifically associated with the receptor, e.g., conformational change, phosphorylation, nature or quantity of proteins associated with the receptor, or change in genetic expression mediated by or associated with the receptor.

“Small molecules” are provided for the treatment of physiology and disorders of tumors and cancers. “Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described (see, e.g., Casset, et al. (2003) Biochem. Biophys. Res. Commun. 307:198-205; Muyldermnans (2001) J. Biotechnol. 74:277-302; Li (2000) Nat. Biotechnol. 18:1251-1256; Apostolopoulos, et al. (2002) Curr. Med. Chem. 9:411-420; Monfardini, et al. (2002) Curr. Pharm. Des. 8:2185-2199; Domingues, et al. (1999) Nat. Struct. Biol. 6:652-656; Sato and Sone (2003) Biochem. J. 371:603-608; U.S. Pat. No. 6,326,482 issued to Stewart, et al).

“Soluble receptor” refers to receptors that are water-soluble and occur, e.g., in extracellular fluids, intracellular fluids, or weakly associated with a membrane. Soluble receptor further refers to receptors that are engineered to be water soluble.

“Specificity of binding,” “selectivity of binding,” and the like, refer to a binding interaction between a predetermined ligand and a predetermined receptor that enables one to distinguish between the predetermined ligand and other ligands, or between the predetermined receptor and other receptors. “Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, binds to its antigen with an affinity that is at least two fold greater, preferably at least ten times greater, more preferably at least 20-times greater, and most preferably at least 100-times greater than the affinity to any other antigen. In a preferred embodiment the antibody will have an affinity that is greater than about 10⁹ liters/mol (see, e.g., Munsen, et al. (1980) Analyt. Biochem. 107:220-239).

II. General.

The present invention provides methods for the modulation or treatment of a number of immune conditions and disorders, e.g., psoriasis, rheumatoid arthritis, Crohn's disease (CD), and certain cancers. Provided are methods for the treatment and diagnosis of disorders characterized by abnormal expression of p28, EBI3, IL-27, and WSX-1/TCCR.

The physiology and immunology of IL-27, IL-27 receptor, and its subunits, is reviewed. In short, IL-27, or one of its subunits, has been found to play a role in interferon-gamma (IFNgamma) response, T cell differentiation, Epstein-Barr virus induced disorders, pregnancy, and ulcerative colitis (but not Crohn's disease),

In detail, IL-27 influences the pathway of T cell differentiation involving TNFalpha-stimulated DCs, where the TNFalpha-stimulated DCs contact naïve T cells and promote differentiation of the naïve T cells to IFNgamma-producing T cells. If IL-27 is present during the contacting of the TNFalpha-stimulated DC to the naïve T cell, this will enhance the T cell's production of IFNgamma. Thus, IL-27 contributes to DC-dependent differentiation of naïve TH1-type T cells. IL-27 also has a role in interferon-beta (IFNbeta) action. EBI3 expression by immature dendritic cells (DCs) and mature DCs is stimulated by a number of cytokines. These cytokines include interferon-beta (IFNbeta), and IFNbeta treatment followed by the combination of CD40L, and IFNgamma (see, e.g., Nagai, et al. (2003) J. Immunol. 171:5233-5243; van Seventer, et al. (2002) J. Neuroimmunol. 133:60-71).

EBI3 appears to have a role in Epstein-Barr virus-induced disorders. EBI3 is expressed with infection of Epstein-Barr virus of B cells, an infection resulting in mononucleosis. EBI3, expressed by Hodgkin lymphoma-derived cell lines and in some nasopharyngeal carcinomas, has been proposed to be used by tumors or viruses to inhibit immune response against tumors associated with Epstein-Barr virus, i.e., certain Hodgkin lymphomas and nasopharyngeal carcinomas. In short, it was proposed that EBI3 has an immunosuppressive or TH2-promoting function (see, e.g., Devergne, et al. (1996) J. Virol. 70:1143-1153; Niedobitek, et al. (2002) J. Pathol. 198:310-316).

IL-27 has a role specific to pregnancy. IL-27 is expressed in the uterus after gestation starts, where expression of this cytokine occurs in uterine NK cells. EBI3, a subunit of IL-27, shows increased expression by the placenta, that is, by differentiated trophoblast cells, and is found in increased amounts in serum during pregnancy (see, e.g., Croy, et al. (2003) Reproduction 126:149-160; Zhang, et al. (2003) Biol. Reproduction 69:404-411; Devergne, et al. (2001)Am. J. Pathol. 159:1763-1776).

An EBI3 knockout mouse (EBI3KO mouse; EBI3^{-/-} mouse) was prepared to determine the consequences on physiology, e.g., of the immune system. The EBI3KO mice showed changes in invariant NK T cells (iNK T cells), and CD4⁺ T cells. The EBI3KO produced decreased numbers of iNK T cells. With the EBI3KO, CD4⁺ T cells from spleen showed more IFNgamma production, upon cell activation, but less IL-4, upon cell activation. These effects indicate that the EBI3KO promotes TH1-type response, and that EBI3 contributes to TH2-type response. The EBI3KO reduced the number of iNK T cells, and also reduced the iNK T cell's ability, on a per cell basis, to produce IL-4. The EBI3KO mice also become resistant to colitis, as demonstrated by studies on oxazolone-induced colitis, a model of TH2-type immune response mediated colitis, though the EBI3KO mice did not resist a model of TH1-type colitis. Similarly, in another study indicating a role for EBI3 in a TH2-type colitis, EBI3 had enhanced expression in active ulcerative colitis, a disorder where a TH2-type response predominates but not in active Crohn's disease, where a TH1-type response can predominate (Christ, et al. (1998) Gastroentrol. 115:307-313; Niedobitek, et al. (2002) J. Pathol. 198:310-316).

WSX-1/TCCR is expressed in CD4⁺ T cells, CD8⁺ T cells, and CD19⁺ B cells (see, e.g., Sprecher, et al. (1998) Biochem. Biophys. Res. Commun. 246:82-90).

The present invention identifies gp130 as a subunit of the IL-27 receptor. gp130 is a receptor subunit that is a shared receptor subunit the IL-6 family of cytokines. Thus, gp130 is a subunit of the receptors for IL-6, leukemia inhibitory factor (LIF), IL-11, oncostatin M, ciliary neuroptrophic factor (CNTF), cardiotrophin-1 (CT-1), cardiotrophin-like cytokine (CLC), and the viral IL-6 homologue. Soluble versions of gp130 have been identified (see, e.g., Hammacher, et al. (1998) J. Biol. Chem. 273:22701-22707; Hammacher, et al. (2000) Biochem. J. 345:25-32; Sanchez-Cuenca, et al. (1999) Immunol. Today 20:57-59; Gadient and Patterson (1999) Stem Cells 17:127-137; Peters, et al. (1996) J. Exp. Med. 183:1399-1406; Muller-Newen (2003) Science STKE 2003, pe40).

The present invention provides methods for the treatment and diagnosis of Crohn's disease. Crohn's disease is a chronic inflammatory disorder that can affect any region of the gastrointestinal tract, e.g., the small intestines or colon. Crohn's disease involves fistula, while another inflammatory disorder of the gut, ulcerative colitis, involves shallow, ulcerative lesions. The pathology of Crohn's disease involves inflammatory cytokines, e.g., IL-1, IL-6, and tumor necrosis factor (TNF). Crohn's disease is distinguished from ulcerative colitis in that Crohn's disease generally involves a TH1-type response with an early increase in IFN, IL-2, and IL-12, with later increases in TNFalpha and IL-18.

Contrasting with Crohn's disease, in ulcerative colitis there is increased expression of IL-5, IL-6, IL-10, and IL-13, and here, the cytokine pattern resembles a variation of the TH2-type response. Crohn's disease and ulcerative colitis are further distinguished in that in the former, T cells in mucosal lesions resist apoptosis, while in the latter, T cells in mucosal lesions are more susceptible to Fas-mediated apoptosis. Mutations in the NOD2 gene are associated with human Crohn's disease, while “leucocyte antigen region genes” and the MUC3 gene are associated with human ulcerative colitis. Differences in the mechanisms of TH1-type and TH2-type inflammatory bowel disorders is highlighted by the fact that both TH-1 type and TH-2 type mouse models are available. For example, mice given CD45RB^{high}CD4⁺ T cells develop a TH1-cell-mediated disorder resembling human Crohn's disease. A TH2-driven model of inflammatory bowel disease is able with use of a TCRalpha knockout mouse (see, e.g., Ardizzone and Porro (2002) J. Int. Med. 252:475-496; Madsen (2002) Gastroentrol. 123:2140-2144; Bouma and STrober (2003) Nat. Rev. Immunol. 3:521-533; Stallmach, et al. (1998) Immunol. Today19:438-441; Yamamoto, et al. (2000) J. Immunol. 164:4878-4882; Targan, et al. (1997) New Engl. J Med. 337:1029-1035; Simpson, et al. (1998) J. Exp. Med. 187:1225-1234; Beutler (2001) Immunity 15:5-14).

The present invention provides methods for the treatment and diagnosis of psoriasis and other inflammatory disorders of the skin, e.g., contact hypersensitivity. Psoriasis, a common disorder affecting about 2% of the world's population, involves scaling of the skin and pustular lesions. Of the psoriasis patients in the United States, about one million require ultraviolet or immunosuppressive therapy. About 10% of patients with psoriasis also develop psoriatic arthritis, a debilitating condition. Psoriasis involves hyperproliferation of keratinocytes and infiltration of white blood cells in the skin. The inflammation of psoriasis is mediated by, e.g., T cells, monocytes and macrophages, neutrophils, mast cells, and antigen presenting cells (APCs) such as dendritic cells and Langerhans cells (see, e.g., Yu, et al. (2002) Dermatol. 204:94-99; Jiang, et al. (2001) Int. J. Dermatol. 40:699-703).

Keratinocyte hyperproliferation arises, in part, from inappropriate expression of IL-2, IFNgamma, TNFbeta, IL-5, and other cytokines. Innate response, e.g., involving bacterial lipopolysaccharide (LPS; glycolipid), has been implicated as part of the etiology of psoriasis (see, e.g., Bos and De Rie (1999) Immunology Today 20:40-46; Ellis, et al. (2001) New Engl. J. Med. 345:248-255; Bhalerao and Bowcock (1998) Human Mol. Genetics 7:1537-1545; van de Kerkhof(2000) Clin. Exp. Dermatol. 25:165; Tanaka, et al. (2000) Brit. J. Dermatol. 143:728-732; Nickoloff(1999) J. Clin. Invest. 104:1161-1164; Curry, et al. (2003) Arch. Pathol. Lab. Med. 127:178-186; Travers, et al. (1999) J. Clin. Invest. 104:1181-1189; Greaves and Weinstein (1995) New Engl. J. Med. 332:581-588; Robert and Kupper (1999) New Engl. J. Med. 341:1817-1828; Bos and De Rie (1999) Immunol. Today 20:40-46), Shimizu, et al. (2002) Histochem. Cell Biol. 118:251-257, Gottleib, et al. (1995) Nature Med. 1:442-447, Abrams, et al. (2000) J. Exp. Med. 192:681-693; Yu, et al. (2002) Dermatology 204:94-99). Psoriatic arthritis, atopic dermatitis, and asthma are associated with psoriasis (McInnes, et al. (2001) J. Immunol. 176:4075-4082; Welp, et al. (1989) Hautarzt 40:496-500).

The present invention provides methods for the treatment and diagnosis of atherosclerosis and other aspects of cardiovascular disease. Immune cells, e.g., mast cells, dendritic cells, neutrophils, monocytes, and macrophages, contribute to the pathology of atherosclerosis. Tumor necrosis factor, interleukin-1, and other cytokines, have been linked with the etiology of, e.g., atherosclerosis, cardiovascular disease, and stroke (see, e.g., Huang, et al. (2002) Cardiovasc. Res. 55:150-160; Kelley, et al. (2000) Mol. Med. Today 6:304-308; Aicher, et al. (2003) Circulation 107:604-611; Ozmen, et al. (2002) Histol. Histopathol. 17:223-237; Wanders, et al. (1994) Transpl. Int. 7Suppl. 1:S371-S375; Hallenbeck (2002) Nature Med. 8:1363-1368; Young, et al. (2002) Thromb. Haemost. 88:554-567; Loppnow, et al. (2001) Shock 1:3-9).

Additionally, the present invention provides methods of the treatment and diagnosis of arthritis, e.g., rheumatoid arthritis, psoriatic arthritis, juvenile rheumatoid arthritis, osteoarthritis, and ankylosing spondylitis. Rheumatoid arthritis (RA) is a chronic, destructive disease of the joints, characterized by inflammation and synovial hyperplasia. The disease cannot be cured and results in disablement. CD4⁺ T cells infiltrate the joints and stimulate the production of IL-1, IL-6, and TNFalpha. The produced cytokines stimulate fibroblasts, osteoclasts, and chondrocytes to release proteinases which, in turn, degrade cartilage of the joints. The mast cell is a key immune cell involved in RA pathology. Mast cells produce tumor necrosis factor-alpha (TNFalpha) which initiates an inflammatory cascade that promotes expression of IL-1 and IL-6. The mast cell also activates proteases which degrade the cartilage matrix. Mouse models of arthritis are available, e.g., collagen-induced arthritis (CIA), TNF overexpressing mice, and IL-1alpha overexpressing mice (Choy and Panayi (2001) New Engl. J. Med. 344:907-916; Woolley (2003) New Engl. J. Med. 348:1709-1711; Niki, et al. (2001) J. Clin. Invest. 107:1127-1135; Feldmann and Maini (2001) Annu. Rev. Immunol. 19:163-196).

The present invention provides methods for the treatment and diagnosis of asthma and allergies. Infection with a helminth, e.g., Aspergillus or Nippostrongylus, is associated in humans with asthma and allergies. Moreover, infection with Aspergillus or Nippostrongylus, or treatment with a helminth antigen, has been used in model studies of asthma and allergies. Immune response to helminth allergens can occur in phases, e.g., an early phase or a late phase (see, e.g., Hurst, et al. (2001) J. Immunol. 166:4922-4930; Hurst, et al. (2002) J. Immunol. 169:443-453; Mehrad, et al. (1999) J. Immunol. 162:1633-1640; Soubani and Chandrasekar (2002) Chest 121:1988-1999; Schuh, et al. (2002) FASEB J. 16:1313-1315; Greenberger (2003) Front Biosci. 8:s119-s127; Gibson, et al. (2003) Eur. Respir. J. 21:582-588; Black, et al. (2001) J. Appl. Physiol. 90:571-578; Palmer, et al. (2002) Am. J. Respir. Crit. Care Med. 165:1489-1493; Zou, et al. (2002) Genome Biol. 3:20.1-20.13; Abraham, et al. (1999) Am. J. Respir. Crit. Care Med. 159:1205-1214; Jones, et al. (1998) Can. J. Physiol. Pharmacol. 76:210-217; Wright, et al. (1999) J. Pharmacol. Exp. Therapeutics 289:1007-1014; D'Brot, et al. (1989) Am. Rev. Respir. Dis. 139:915-920).

The present invention also contemplates methods of treatment and diagnosis of pulmonary disorders, including those involving airway hyperreactivity, e.g., by treating with an antagonist of IL-27. Airway hyperreactivity, also known as airway hyperresponsiveness, which involves inappropriate airway narrowing in response to a stimulus, is a characteristic of various disorders of the airways, e.g., asthma, allergic rhinitis, bronchitis, bronchiolitis, and possibly chronic obstructive pulmonary disorder (COPD). Hyperreactivity can be triggered by, e.g., respiratory infections, smoke, and respiratory allergens. Asthma, a chronic disorder that can be fatal, affects about one in seven children in the United States, and accounts for over 15% of pediatric emergencies. The symptoms involve shortness of breath, and mucus hypersecretion (see, e.g., Crain, et al. (1995) Arch. Pediatr. Adolesc. Med. 149:893-901; Grunig, et al. (1998) Science 282:2261-2263; Crystal, et al. (eds.) (1997) The Lung, Vols. 1-2, 2^{nd} ed., Lippincott-Raven, Phila, Pa.; Holgate, et al. (2001) Allergy, 2^{nd} ed., Mosby, N.Y.; Marone (1998) Immunol. Today 19:5-9; Barnes and Lemanske (2001) New Engl. J. Med. 344:350-362).

Airway hyperreactivity is characterized by infiltration by T cells, eosinophils, mast cells, neutrophils, and antigen presenting cells (APCs), in the airways. The APCs of the lung include DCs, B cells, and alveolar macrophages, each of which can express cytokines and contribute to airway hyperreactivity (see, e.g., Lawrence, et al. (1998) J. Pharm. Exp. Thera. 284:222-227; Alexis, et al. (2001) Am. J. Physiol. Lung Cell Mol. Physiol. 280:L369-L375; Akabari, et al. (2002) Nature Medicine 8:1024-1032; MacLean, et al. (1999) Am. J. Respir. Cell Mol. Biol. 20:379-387; Hamelmann, et al. (1999) Am. J. Respir. Cell Mol. Biol. 21:480-489; Gonzales, et al. (2000) Annals Internal Medicine 133:981-991; Li, et al. (2002) Pulmonary Pharmacol. Therapeutics 15:409-416; Zimmermann, t al. (2003) J. Allergy Clin. Immunol. 111:227-242; Riffo-Vasquez and Spina (2002) Pharmacol. Therapeutics 94:185-211).

COPD is a disorder involving bronchiolar infiltration with macrophages, neutrophils, and T cells, e.g., CD8⁺ T cells. COPD, the fourth leading cause of death in North America, is characterized by thickening of airway smooth muscle and inflammation of the airways. This response appears to be due to the infiltration of monocytes, macrophages, CD4⁺ T cells, CD8⁺ T cells, and neutrophils to the lungs. Alveolar macrophages, elevated in COPD, express cytokines that, in turn, promote inflammation and increase in immune cell activation. COPD involves chronic bronchitis and emphysema. Emphysema is characterized by permanent destruction of the parenchyma, airspaces distal to the terminal bronchioli (see, e.g., Hautamaki, et al. (1997) Science 277:2002-2004; Barnes (2000) Chest 117: 10S-14S; Barnes (2003) Annu. Rev. Med. 54:113-129; Jeffery (1998) Thorax 53:129-136; Barnes (2000) New Engl. J. Med. 343:269-280). Cancer treatment and diagnostic methods are encompassed by the present invention. Note that IL-27 has been shown to treat tumors in mice (Hisada, et al. (2004) Cancer Res. 64:1152-1156). The present invention provides methods of using IL-27 to increase production of TNFalpha, IL-1alpha, and OX40, cytokines that have been implicated with proper immune response against tumors and with tumor regression. The present invention provides methods to treat cancer by using IL-27 to stimulate production of anti-tumor immune response involving cytokines such as TNFalpha, IL-1alpha, IL-1beta, and OX40. Tumors are often infiltrated by CD4⁺ T cells and CD8⁺ T cells. Higher infiltration of a tumor with T cells is sometimes associated with better prognosis for the patient, e.g., in the case of melanoma and colorectal cancer. A problem with immune response to tumors is that the T cells can be incompletely activated, anergic, or inactivated (Dalerba, et al. (2003) Crit. Revs. Oncology Hematology 46:33-57; Ladanyi, et al. (2004) Clin. Cancer Res. 10:521-530; Toomey, et al. (1999) Immunol. Invest. 28:29-41).

IL-1alpha, IL-1beta, and TNFalpha have anti-tumor effects, resulting in enhanced immune response against the tumor. IL-lalpha is found to be expressed by a number of tumor types. The anti-tumor effects of TNFalpha, for example, result from direct cytotoxicity to the tumor, but also via activation of macrophages, CD8⁺ T cells, and neutrophils. In contrast, under certain conditions IL-1 and TNFalpha can have a pro-tumor effect. IL-1 can induce secretion of factors that promote tumor growth and invasiveness. Production of IL-1 can resulting autocrine activation, increasing invasiveness. TNFalpha, IL-1alpha, and IL-1beta can stimulate growth of certain tumors, e.g., ovarian tumors (see, e.g., Chen, et al. (1998) Cancer Res. 58:3668-3676; Woods, et al. (1998) Cancer Res. 58:3132-3141; Apte and Voronov (2002) Sem. Cancer Biol. 12:277-290; Woodward, et al. (2002) Invest. Ophthalmol. Vis. Sci. 43:3144-3152;; Smith, et al. (1990) Cancer Res. 50:3146-3153; Wu, et al. (1993) Cancer Res. 53:1939-1944; Noorda, et al. (2003) Cancer 98:1483-1490; Bazzoni, et al. (2001) Cancer Res. 61:1050-1057; Kamada, et al. (2000) Cancer Res. 60:6416-6420; Kaneda, et al. (1998) Cancer Res. 58:290-295; Gnant, et al. (1999) Cancer Res. 59:4668-4674; Suganuma, et al. (1999) Cancer Res. 59:4516-4518).

OX40 is a ligand, whereas OX40R is the corresponding receptor. OX40/OX40R mediated signaling plays a part in anti-tumor response. OX40 and OX40R are upregulated in T cells that infiltrate tumors, but are not upregulated in peripheral blood T cells. Triggering OX40/OX40R signaling by administering OX40 ligand can result in rejection of various tumors. Human breast cancer and melanomas have been found to contain OX40-expressing T cells, again implicating OX40/OX40R in anti-tumor response (see, e.g., Morris, et al. (2001) Breast Cancer Res. Treat. 67:71-80; Hurwitz, et al. (2000) Curr. Opin. Immunol. 12:589-596; Ladany, et al. (2004) Clin. Cancer Res. 10:521-530).

With respect to cancer, various methods of modulating immune response for the treatment of cancers, tumors, metastasis, and angiogenesis, are available. These methods include treatment with cytokines or anti-cytokine antibodies, such as IL-2, IL-12, tumor necrosis factor-alpha (TNFalpha), IFNgamma, granulocyte macrophage-colony stimulating factor (GM-CSF), and transforming growth factor (TGF). Where a cancer cell can produces a cytokine that enhance its own growth or its own survival, an anti-cytokine antibody may be an appropriate therapeutic agent (see, e.g., Ramirez-Montagut, et al. (2003) Oncogene 22:3180-3187; Braun, et al. (2000) J. Immunol. 164:4025-4031; Shaw, et al. (1998) J. Immunol. 161:2817-2824; Coussens and Werb (2002) Nature 420:860-867; Baxevanis, et al. (2000) J. Immunol. 164:3902-3912; Shimizu, et al. (1999) J. Immunol. 163:5211-5218; Belardelli and Ferrantini (2002) TRENDS Immunol. 23:201-208; Seki, et al. (2002) J. Immunol. 168:3484-3492; Casares, et al. (2003) J. Immunol. 171:5931-5939; Oft, et al. (2002) Nature Cell Biol. 4:487494).

III. Agonists, Antagonists, and Binding Compositions.

The present invention provides methods of using agonists and antagonist of IL-27. An agonist of IL-27 encompasses, e.g., IL-27, an IL-27 variant, mutein, hyperkine, or peptide mimetic thereto, agonistic antibodies to WSX-1/TCCR or gp130, and nucleic acids encoding these agonists. Antagonists of IL-27 include, e.g., antibodies to IL-27, antibodies to p28 or EBI3, blocking antibodies to WSX-1/TCCR or gp130, a soluble receptor based on the extracellular region of a subunit of WSX-1/TCCR or gp130 , peptide mimetics thereto, and nucleic acids encoding these antagonists. Anti-idiotypic antibodies may also be used.

The present invention provides methods of using agonists and antagonists of p28, agonists and antagonists of the complex of p28 and EBI3, agonists and antagonists of WSX-1/TCCR, agonists and antagonists of gp130, and agonists and antagonists of the complex of WSX-1/TCCR and gp130 .

An IL-27 hyperkine encompasses, e.g., a fusion protein comprising the polypeptide sequence of p28 and EBI3, where p28 and EBI3 occur in one continous polypeptide chain. The sequences of p28 and EBI3 may be in either order in the continuous polypeptide chain. The fusion protein may contain a linker sequence, residing in between the sequences of p28 and EBI3, in one continuous polypeptide chain.

Regions of increased antigenicity that can be used for antibody generation can readily be found with a Parker plot using Vector NTI® Suite (Informax, Inc, Bethesda, Md.).

Antibodies to p28, EBI3, WSX-1/TCCR, and gp130 are available (see, e.g., Pflanz, et al. (2004) J. Immunol. 172:2225-2231; Larousserie, et al. (2004) J. Pathol. 202:164-171; Devergne, et al. (2001)Am. J. Pathol. 159:1763-1776; Autissier, et al. (1998) Int. Immunol. 10:1881-1889). Also contemplated are antibodies that specifically bind the complex of p28 and EBI3, and antibodies that specifically bind to the complex of WSX-1/TCCR and gp130.

Also provided are soluble receptors corresponding to an extracellular domain of WSX-1/TCCR and gp130 . The extracellular domain of mature human WSX-1/TCCR comprises amino acids 33 to 514 of the amino acid sequence of GenBank BC028003 or NM_{—}004843. This extracellular domain includes a classical cytokine binding domain, and also three fibronectin (FN) domains. The invention contemplates a soluble receptor comprising the cytokine binding domain and none, one, or, or three of the FN domains (Sprecher, et al., supra). Soluble gp130 is available (see, e.g., Hui, et al. (2000) Cytokine 12:151-155).

Receptors based on these extracellular regions are not limited by these exact N-terminal and C-terminal amino acids, but may be longer or shorter, e.g., by one, two, three, or more amino acids, as long as the ligand binding properties are substantially maintained. Fusion proteins based on the soluble receptors are also contemplated, e.g., for facilitating purification or stability or for providing a functional domain, e.g., a toxic polypeptide.

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (I999) J. Biol. Chem. 274:27371-27378; Baca, et al. (1997) J. Biol. Chem. 272:10678-10684; Chothia, et al. (1989) Nature 342:877-883; Foote and Winter (1992) J. Mol. Biol. 224:487-499; U.S. Pat. No. 6,329,511 issued to Vasquez, et al.). Muteins and variants of antibodies and soluble receptors are contemplated, e.g., pegylation or mutagenesis to remove or replace deamidating Asn residues.

Purification of antigen is not necessary for the generation of antibodies. Immunization can be performed by DNA vector immunization, see, e.g., Wang, et al. (1997) Virology 228:278-284. Alternatively, animals can be immunized with cells bearing the antigen of interest. Splenocytes can then be isolated from the immunized animals, and the splenocytes can fused with a myeloma cell line to produce a hybridoma (Meyaard, et al. (1997) Immunity 7:283-290; Wright, et al. (2000) Immunity 13:233-242; Preston, et al. (1997) Eur. J. Immunol. 27:1911-1918). Resultant hybridomas can be screened for production of the desired antibody by functional assays or biological assays, that is, assays not dependent on possession of the purified antigen. Immunization with cells may prove superior for antibody generation than immunization with purified antigen (Kaithamana, et al. (1999) J. Immunol. 163:5157-5164).

Antibodies will usually bind with at least a K_{D} of about 10⁻³ M, more usually at least 10⁻⁶ M, typically at least 10⁻⁷ M, more typically at least 10⁻⁸ M, preferably at least about 10⁻⁹ M, and more preferably at least 10⁻¹⁰ M, and most preferably at least 10⁻¹¹ M (see, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s).

Soluble receptors comprising the extracellular domains of WSX-1/TCCR or gp130 receptor polypeptides are provided. Soluble receptors can be prepared and used according to standard methods (see, e.g., Jones, et al. (2002) Biochim. Biophys. Acta 1592:251-263; Prudhomme, et al. (2001) Expert Opinion Biol. Ther. 1:359-373; Fernandez-Botran (1999) Crit. Rev. Clin. Lab Sci. 36:165-224). Also provided are compositions for siRNA interference (see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189).

IV. Therapeutic Compositions, Methods.

The present invention provides methods for treating and diagnosing psoriasis, Crohn's disease, rheumatoid arthritis, and cancer.

To prepare pharmaceutical or sterile compositions including an agonist or antagonist of IL-27, the reagent is mixed with a pharmaceutically acceptable carrier or excipient. Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman 's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Antibodies, antibody fragments, and cytokines can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 ag/kg, more typically at least 10 μg/kg, most typically at least 100 μg/kg, preferably at least 0.2 μg/kg, more preferably at least 1.0 mg/kg, most preferably at least 2.0 mg/kg, optimally at least 10 mg/kg, more optimally at least 25 mg/kg, and most optimally at least 50 mg/kg (see, e.g., Yang, et al. (2003) New Engl. J. Med. 349:427-434; Herold, et al. (2002) New Engl. J. Med. 346:1692-1698; Liu, et al. (1999) J. Neurol. Neurosurg. Psych. 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg body weight basis. The desired plasma concentration of a small molecule therapeutic is about the same as for an antibody, on a moles/kg body weight basis.

An effective amount for a particular patient may vary depending on factors such as the condition being treated, the overall health of the patient, the method route and dose of administration and the severity of side affects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

Typical veterinary, experimental, or research subjects include monkeys, dogs, cats, rats, mice, rabbits, guinea pigs, horses, and humans.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

Methods for co-administration or treatment with a second therapeutic agent, e.g., a cytokine, steroid, chemotherapeutic agent, antibiotic, or radiation, are well known in the art (see, e.g., Hardman, et al. (eds.) (2001) Goodman and Gilman 's The Pharmacological Basis of Therapeutics, 10^{th} ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic will decrease the symptoms typically by at least 10%; usually by at least 20%; preferably at least about 30%; more preferably at least 40%, and most preferably by at least 50%.

The route of administration is by, e.g., topical or cutaneous application, injection or infusion by intravenous, intraperitoneal, intracerebral, intramuscular, intraocular, intraarterial, intracerebrospinal, intralesional, or pulmonary routes, or by sustained release systems or an implant (see, e.g., Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. Natl. Acad. Sci. USA 82:3688-3692; Hwang, et al. (1980) Proc. Natl. Acad. Sci. USA 77:4030-4034; U.S. Pat. Nos. 6,350466 and 6,316,024).

The present invention provides methods of treating or diagnosing a proliferative

condition or disorder, e.g., cancer of the uterus, cervix, breast, prostate, testes, penis, gastrointestinal tract, e.g., esophagus, oropharynx, stomach, small or large intestines, colon, or rectum, kidney, renal cell, bladder, bone, bone marrow, skin, head or neck, skin, liver, gall bladder, heart, lung, pancreas, salivary gland, adrenal gland, thyroid, brain, ganglia, central nervous system (CNS) and peripheral nervous system (PNS), and immune system, e.g., spleen or thymus. The present invention provides methods of treating, e.g., immunogenic tumors, non-immunogenetic tumors, dormant tumors, virus-induced cancers, e.g., epithelial cell cancers, endothelial cell cancers, squamous cell carcinomas, papillomavirus, adenocarcinomas, lymphomas, carcinomas, melanomas, leukemias, myelomas, sarcomas, teratocarcinomas, chemically-induced cancers, metastasis, and angiogenesis. The invention also contemplates reducing tolerance to a tumor cell or cancer cell antigen, e.g., by modulating activity of a regulatory T cell (Treg) (see, e.g., Ramirez-Montagut, et al. (2003) Oncogene 22:3180-3187; Sawaya, et al. (2003) New Engl. J. Med. 349:1501-1509; Farrar, et al. (1999) J. Immunol. 162:2842-2849; Le, et al. (2001) J. Immunol. 167:6765-6772; Cannistra and Niloff (1996) New Engl. J. Med. 334:1030-1038; Osborne (1998) New Engl. J. Med. 339:1609-1618; Lynch and Chapelle (2003) New Engl. J. Med. 348:919-932; Enzinger and Mayer (2003) New Engl. J. Med. 349:2241-2252; Forastiere, et al. (2001) New Engl. J. Med. 345:1890-1900; Izbicki, et al. (1997) New Engl. J. Med. 337:1188-1194; Holland, et al. (eds.) (1996) Cancer Medicine Encyclopedia of Cancer, 4^{th} ed., Academic Press, San Diego, Calif.).

The present invention provides methods for treating a proliferative condition, cancer, tumor, or precancerous condition such as a dysplasia, with an agonist or antagonist of IL-27, with at least one additional therapeutic or diagnostic agent. One or more additional therapeutic or diagnostic agents can be selected from, e.g., a cytokine or cytokine antagonist, such as interferon-alpha, or anti-epidermal growth factor receptor, doxorubicin, epirubicin, an anti-folate, e.g., methotrexate or fluoruracil, irinotecan, cyclophosphamide, radiotherapy, hormone or anti-hormone therapy, e.g., androgen, estrogen, anti-estrogen, flutamide, or diethylstilbestrol, surgery, tamoxifen, ifosfamide, mitolactol, an alkylating agent, e.g., melphalan or cis-platin, etoposide, vinorelbine, vinblastine, vindesine, a glucocorticoid, a histamine receptor antagonist, an angiogenesis inhibitor, radiation, a radiation sensitizer, anthracycline, vinca alkaloid, taxane, e.g., paclitaxel and docetaxel, a cell cycle inhibitor, e.g., a cyclin-dependent kinase inhibitor, a monoclonal antibody, a complex of monoclonal antibody and toxin, a T cell adjuvant, bone marrow transplant, or antigen presenting cells, e.g., dendritic cell therapy. Vaccines can be provided, e.g., as a soluble protein or as a nucleic acid encoding the protein (see, e.g., Le, et al. (2001) J. Immunol. 167:6765-6772; Greco and Zellefsky (eds.) (2000) Radiotherapy of Prostate Cancer, Harwood Academic, Amsterdam; Shapiro and Recht (2001) New Engl. J. Med. 344:1997-2008; Hortobagyi (1998) New Engl. J. Med. 339:974-984; Catalona (1994) New Engl. J. Med. 331:996-1004; Naylor and Hadden (2003) Int. Immunopharmacol. 3:1205-1215; The Int. Adjuvant Lung Cancer Trial Collaborative Group (2004) New Engl. J. Med. 350:351-360; Slamon, et al. (2001) New Engl. J. Med. 344:783-792; Kudelka, et al. (1998) New Engl. J. Med. 338:991-992; van Netten, et al. (1996) New Engl. J. Med. 334:920-921).

V. Kits and Diagnostic Reagents.

Diagnostic methods for inflammatory disorders, e.g., psoriasis, Crohn's disease, rheumatoid arthritis, asthma or allergy, atherosclerosis, and cancers, based on antibodies, nucleic acid hybridization, and the PCR method, are available.

This invention provides polypeptides of IL-27, fragments thereof, nucleic acids of IL-27, and fragments thereof, in a diagnostic kit, e.g., for the diagnosis of viral disorders, including of influenza A, and viral disorders of the respiratory tract and of mucosal tissues. Also provided are binding compositions, including antibodies or antibody fragments, for the detection of IL-27, and metabolites and breakdown products thereof. Typically, the kit will have a compartment containing either a IL-27 polypeptide, or an antigenic fragment thereof, a binding composition thereto, or a nucleic acid, such as a nucleic acid probe, primer, or molecular beacon (see, e.g., Rajendran, et al. (2003) Nucleic Acids Res. 31:5700-5713; Cockerill (2003) Arch. Pathol. Lab. Med. 127:1112-1120; Zammatteo, et al. (2002) Biotech. Annu. Rev. 8:85-101; Klein (2002) Trends Mol. Med. 8:257-260).

A method of diagnosis can comprise contacting a sample from a subject, e.g., a test subject, with a binding composition that specifically binds to a polypeptide or nucleic acid of IL-27 or IL-27 receptor. The method can further comprise contacting a sample from a control subject, normal subject, or normal tissue or fluid from the test subject, with the binding composition. Moreover, the method can additionally comprise comparing the specific binding of the composition to the test subject with the specific binding of the composition to the normal subject, control subject, or normal tissue or fluid from the test subject. Expression or activity of a test sample or test subject can be compared with that from a control sample or control subject. A control sample can comprise, e.g., a sample of non-affected or non-inflamed tissue in a patient suffering from an immune disorder. Expression or activity from a control subject or control sample can be provided as a predetermined value, e.g., acquired from a statistically appropriate group of control subjects.

The kit may comprise, e.g., a reagent and a compartment, a reagent and instructions for use, or a reagent with a compartment and instructions for use. The reagent may comprise an agonist or antagonist of IL-27, or an antigenic fragment thereof, a binding composition, or a nucleic acid in a sense and/or anti-sense orientation. A kit for determining the binding of a test compound, e.g., acquired from a biological sample or from a chemical library, can comprise a control compound, a labeled compound, and a method for separating free labeled compound from bound labeled compound. The control compound can comprise a segment of the polypeptide of IL-27 or IL-27 receptor or a nucleic acid encoding IL-27 or IL-27 receptor. The segment can comprise zero, one, two, or more antigenic fragments.

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, fluorescent dyes, electron-dense reagents, substrates, epitope tags, or enzymes, e.g., as used in enzyme-linked immunoassays, or fluorettes (Rozinov and Nolan (1998) Chem. Biol. 5:713-728).

Diagnostic assays can be used with biological matrices such as live cells, cell extracts, cell lysates, fixed cells, cell cultures, bodily fluids, or forensic samples. Conjugated antibodies useful for diagnostic or kit purposes, include antibodies coupled to dyes, isotopes, enzymes, and metals, see, e.g., Le Doussal, et al. (1991) New Engl. J. Med. 146:169-175; Gibellini, et al. (1998) J. Immunol. 160:3891-3898; Hsing and Bishop (1999) New Engl. J. Med. 162:2804-2811; Everts, et al. (2002) New Engl. J. Med. 168:883-889. Various assay formats exist, such as radioimmunoassays (RIA), ELISA, and lab on a chip (U.S. Pat. Nos. 6,176,962 and 6,517,234).

Gene expression data is useful tool in the diagnosis and treatment of diseases and pathological conditions (see, e.g., Li and Wong (2001) Genome Informatics 12:3-13; Lockhart, et al. (1996) Nature Biotechnol. 14:1675-1680; Homey, et al. (2000) J. Immunol. 164:3465-3470; Debets, et al. (2000) J. Immunol. 165:4950-4956).

VI. Uses.

The present invention provides methods using agonists and antagonists of IL-27 and IL-27 receptor for the diagnosis, prevention, and treatment of immune and inflammatory disorders, including disorders of the skin, gastrointestinal tract, joints, and vascular system, such as psoriasis, Crohn's disease, rheumatoid arthritis, asthma, allergies, COPD, airway hyperreactivity, and atherosclerosis. The present invention also encompasses methods of treating or enhancing inappropriate or inadequate immune response during cancers, e.g., breast cancer and melanoma. Provided are methods to modulate immune response to, or response of a cell during, e.g., psoriasis, Crohn's disease, rheumatoid arthritis, asthma, allergies, atherosclerosis, and cancer, by administering an agonist of IL-27 or an antagonist of IL-27, where administration is to, e.g., a cell, biological fluid, tissue, organ, animal subject, or human subject.

A number of biomarkers and methods for scoring inflammatory disorders, e.g., psoriasis, Crohn's disease, and rheumatoid arthritis are available (see, e.g., Bresnihan (2003) Arthritis Res. Ther. 5:271-278; Bamero and Delmas (2003) Curr. Opin. Rheumatol. 15:641-646; Gionchetti, et al. (2003) Dig. Dis. 21:157-167; Wiik (2002) Autoimmune Rev. 1:67-72; Sostegni, et al. (2003) Aliment Pharmacol. Ther. 17 (Suppl.2): 11-17).

Biomarkers and methods for scoring cancer are also described (see, e.g., Alison (ed.) (2001) The Cancer Handbook, Grove's Dictionaries, Inc., St. Louis, Mo.; Oldham (ed.) (1998) Principles of Cancer Biotherapy, 3^{rd}. ed., Kluwer Academic Publ., Hingham, Mass.; Thompson, et al. (eds.) (2001) Textbook of Melanoma, Martin Dunitz, Ltd., London, UK; Devita, et al. (eds.) (2001) Cancer: Principles and Practice of Oncology, 6^{th} ed., Lippincott, Phila, Pa.; Holland, et al. (eds.) (2000) Holland-Frei Cancer Medicine, BC Decker, Phila., Pa.; Garrett and Sell (eds.) (1995) Cellular Cancer Markers, Humana Press, Totowa, N.J.; MacKie (1996) Skin Cancer, 2^{nd} ed., Mosby, St. Louis; Moertel (1994) New Engl. J. Med. 330:1136-1142; Engleman (2003) Semin. Oncol. 30(3 Suppl. 8): 23-29; Mohr, et al. (2003) Onkologie 26:227-233).

The broad scope of this invention is best understood with reference to the following examples, which are not intended to limit the inventions to the specific embodiments.

### EXAMPLES

I. General Methods.

Standard methods in biochemistry and molecular biology are described (see, e.g., Maniatis, et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Methods for the production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protcols in Immunology, Vol. 4, John Wiley, Inc., New York).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (see, e.g., Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology:Text and Atlas, McGraw-Hill, New York, N.Y.).

Methods for using animal models, e.g., knockout mice, and cell-based assays for the testing, evaluation, and screening of diagnostic, therapeutic, and pharmaceutical agents are available (see, e.g., Car and Eng (2001) Vet. Pathol. 38:20-30; Kenyon, et al. (2003) Toxicol. Appl. Pharmacol. 186:90-100; Deurloo, et al. (2001) Am. J. Respir. Cell Mol. Biol. 25:751-760; Zuberi, et al. (2000) J. Immunol. 164:2667-2673; Temelkovski, et al. (1998) Thorax 53:849-856; Horrocks, et al. (2003) Curr. Opin. Drug Discov. Devel. 6:570-575; Johnston, et al. (2002) Drug Discov. Today 7:353-363).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

II. Expression of Subunits of IL-27 and of IL-27 Receptor.

Expression of the subunits of IL-27, i.e., the p28 subunit and the EBI3 subunit, and of the subunits of IL-27 receptor, i.e., the WSX-1/TCCR subunit and the gp130 subunit, were determined by Taqman® real time PCR analysis (Table 1). The results demonstrate the association of increased expression of p28, EBI3, and WSX-1/TCCR with psoriasis; association of enhanced expression of EBI3, WSX-1/TCCR, and gp130 with Crohn's disease; and correlation of increased expression of EBI3 and WSX/TCCR with rheumatoid arthritis. Also shown are associations of increased expression with breast cancer, melanoma, colon carcinoma, and atherosclerosis (Table 1).

TABLE 1
Expression of IL-27 subunits and IL-27 subunits, with analysis by
Taqman ® real time PCR analysis, relative to
expression of ubiquitin (1.0).
p28 subunit

| | |
|---|---|
| normal skin, human | 55.7 |
| psoriasis skin, human | 124.6 |
| Human breast, adjacent to non-infiltrating intraductal carcinoma | 18.5 |
| Human breast, non-infiltrating intraductal carcinoma | 35.4 |
| Human breast, adjacent to infiltrating intraductal carcinoma | 22.5 |
| Human breast, infiltrating intraductal carcinoma | 41.4 |
| Monkey cynomolgus macacque lung control | 0.13 |
| Monkey cynomolgus macacque lung, Ascaris 4 hour anterior | 3.3 |
| Monkey cynomolgus macacque lung, Ascaris 4 hour anterior | 1.0 |
| Mouse control C57BL/6 rag-1 lung | 0.0 |
| Mouse BALB/C lung control | 38.9 |
| Mouse C57BL/6 lung asthma model, Aspergillus challenge | 739.8 |
| (intranasal) | |

EBI3

| | |
|---|---|
| normal skin, human | 6.9 |
| skin psoriasis, human | 18.1 |
| normal colon, human | 11.8 |
| Crohn's colon, human | 127.0 |
| normal synovia, human | 7.1 |
| rheumatoid arthritis synovia, human | 16.9 |

| Cancerous tissue | | Adjacent non-cancerous tissue | |
|---|---|---|---|
| Breast carcinoma noninfiltrating | 40.2 | Breast adjacent 6283 | 16.8 |
| intraductal 6283 | | | |
| Breast carcinoma noninfiltrating | 30.7 | Breast adjacent 8946 | 20.2 |
| intraductal 8946 | | | |
| Breast infiltrating duct 7460 | 40.5 | Breast adjacent 7460 | 28.1 |
| Breast carcinoma infiltrating | 40.6 | Breast adjacent 6613 | 29.6 |
| duct 6613 | | | |
| Breast carcinoma infiltrating | 48.7 | Breast adjacent 7667 | 15.2 |
| duct 7667 | | | |
| Breast carcinoma infiltrating | 35.8 | Breast adjacent 8707 | 17.3 |
| duct 8707 | | | |

WSX-1/TCCR

| | |
|---|---|
| normal skin, human | 0.5 |
| psoriasis skin, human | 46.9 |
| normal colon, human | 1.0 |
| Crohn's colon, human | 77.1 |
| normal synovia, human | 32.7 |
| rheumatoid arthritis synovia, human | 105.0 |
| fibroblast cell line colon, human | 359 |
| colon carcinoma cell line, human | 1120 |
| epithelial cell keratinocyte untreated, human | 50 |
| epithelial cell keratinocyte activated | 150 |
| mast cell resting | 125 |
| mast cell activated | 625 |
| Langerhans cells, resting | 350 |
| Langerhans cells, activated | 480 |
| Monocyte, resting | 200 |
| Monocyte, activated LPS for 1 h. | 600 |

| | | Tissue adjacent | |
|---|---|---|---|
| Cancerous tissue | | to cancerous tissue | |
| Nodular melanoma 11542 | 42.2 | Skin adjacent to 11542 | 11.1 |
| Superficial spreading melanoma | 32.4 | Skin adjacent to 245514 | 13.8 |
| 245514 | | | |
| Superficial spreading melanoma | 14.5 | Skin adjacent to 247034 | 14.7 |
| 247034 | | | |
| Superficial spreading melanoma | 12.5 | Skin adjacent to 247776 | 6.8 |
| 247776 | | | |
| Nodular melanoma 248344 | 64.5 | Skin adjacent to 248344 | 29.8 |

gp130

| | |
|---|---|
| control colon, human | 3.0 |
| Crohn's colon, human | 111.0 |
| aorta untreated mouse C57BL/6 | 0.0 |
| aorta ApoE knockout atherosclerosis model, 5 months old | 74.7 |
| aorta ApoE knockout atherosclerosis model, 12 months old | 0.0 |
| epithelial cell keratinocyte untreated | 200 |
| epithelial cell keratinocyte activated | 375 |
| mast cell resting | 125 |
| mast cell activated | 250 |
| Langerhans cells, resting | 80 |
| Langerhans cells, activated | 250 |
| Monocyte resting | 100 |
| Monocyte, activated LPS for 1 h. | 300 |

Treatment of primary human mast cells, obtained from cord blood, with IL-27 stimulated the expression of a number of genes (Table 2). IL-27 provoked the expression of a number of genes associated with immune disorders such as psoriasis, arthritis, Crohn's disease, asthma, allergies, and airway hyperreactivity (Table 2).

TABLE 2
Real time PCR determination of IL-27-mediated
changes in gene expression by human mast cells. A change in
expression of “1.0” means no detectable change.

| | Change in expression |
|---|---|
| RANKL | 11.1 |
| TNFalpha | 9.8 |
| TEASRL | 9.3 |
| IL-1alpha | 6.4 |
| IL-1beta | 1.8 |
| OX40 | 5.1 |
| APRIL | 2.6 |
| BLYS | 2.3 |
| IL-18 | 1.3 |
| LTalpha | 1.0 |
| LTbeta | 1.0 |
| CD40L | 1.0 |
| CD27L | 0.8 |
| Ubiquitin | 1.0 |

The present invention provides methods for treating psoriasis and other disorders of the skin, e.g., contact hypersensitivity and atopic dermatitis. Psoriasis is associated with increases in expression of, e.g., TNFalpha, IL-1beta, and TEASRL (ligand), TEASR (receptor) (Table 3). Anti-TNF alpha antibody therapy is used in the treatment of psoriasis (see, e.g., Girolomoni, et al. (2002) Curr. Opin. Investig. Drugs. 3:1590-1595; Zabraniecki and Fournie (2001) Joint Bone Spine 68:106-108; Victor and Gottlieb (2002) J. Drugs Dermatol 1:264-275; Reich, et al. (2002) J Invest Dermatol. 118:155-163).

TEASRL (a.k.a. GITRL) is the ligand, while TEASR (a.k.a. GITR) is the receptor, of a signaling pathway involving TEASRL and TEASR. TEASR is also known as, e.g., glucocorticoid-induced tumor necrosis factor receptor (GITR) and TNFRSF 18. TEASR is a member of the tumor necrosis factor receptor superfamily. An agonist of TEASR can result in proliferation of CD4⁺ T cells and CD8⁺ T cells, either by direct stimulation of the CD4⁺ T cell or CD8⁺ T cells, or by breaking suppression mediated by a T regulatory cell (Treg). The Treg can be a CD4⁺ CD25⁺ regulatory T cell (see, e.g., Shimizu, et al. (2002) Nature Immunol. 3:135-142; McHugh, et al. (2002) Immunity 16:311-323).

In view of IL-27's ability to stimulate expression of TEASRL (Table 2), and the association of enhanced TEASRL and TEASR expression with psoriasis (Table 3), the present invention provides an antagonist of IL-27 for the treatment of psoriasis.

TABLE 3
Real time PCR expression of TEASRL (ligand) and TEASR (receptor)
by Taqman ® analysis.

| Expression of TEASRL in human | | Expression of TEASRL in human | |
|---|---|---|---|
| psoriatic skin. | | adjacent normal skin. | |
| Skin, psoriasis PS-017 | 15.0 | Skin, normal PS-017 | 7.9 |
| Skin, psoriasis PS-032 | 25.1 | Skin, normal PS-032 | 1.8 |
| Skin, psoriasis PS-034 | 23.0 | Skin, normal PS-034 | 1.9 |
| Expression of TEASR in human | | Expression of TEASR in human | |
| psoriatic skin. | | adjacent normal skin. | |
| Human skin, psoriasis | 315.4 | Human skin, control | 3.4 |

The present invention provides methods to treat arthritis and psoriatic arthritis. TNFalpha, RANKL, and IL- 1 alpha, expressed at increased levels with IL-27 treatment (Table 2), stimulate the production of osteoclasts, cells that digest and degrade the bone. RANKL is Receptor Activator of Nuclear factor Kappa B Ligand. RANKL expression increases in the joints of human patients with psoriatic arthritis. IL-1alpha and IL-1beta both have roles in the pathology of arthritis. The present invention provides methods for the treatment of arthritis, e.g., rheumatoid arthritis, osteoarthritis, and psoriatic arthritis, by administering an antagonist of IL-27, where the antagonist is expected to reduce expression of TNFalpha and RANKL (Table 2) (see, e.g., Reimold (2002) Curr. Drug Targets Inflamm. Allergy 1:377-392; Girolomoni, et al. (2002) Curr. Opin. Investig. Drugs 3:1590-1595; Ritchlin, et al. (2003) J. Clin. Invest. 111:821-831; Nakashima, et al. (2003) Curr. Opin. Rheumatol. 15:280-287; Williams, et al. (2000) J. Immunol 165:7240-7245; Arend (2001) Semin. Arthritis Rheum. 30 (5 Suppl. 2) 1-6; Arend (2002) Cytokine Growth Factor Revs. 13:323-340).

The present invention provides methods to treat Crohn's disease, e.g., by use of an antagonist of IL-27 to inhibit production of OX40 and/or TNFalpha (Table 2). An antibody to OX40 ameliorated an animal model of Crohn's disease, while increased expression of both OX40 and OX40 ligand (OX40L) was found in the gut of patients with Crohn's disease (see, e.g., Totsuka, et al. (2003) Am. J. Physiol. Gastrointest. Liver Physiol. 284:G595-G603; Souza, et al. (1999) Gut 45:856-863; Stuber, et al. (2000) European J. Clin. Invest. 30:594-599). TNFalpha contributes to Crohn's disease, as an anti-TNFalpha antiobody is used for treating this disorder (see, e.g., Reimold (2002) Curr. Drug Targets Inflamm. Allergy 1:377-392).

The invention provides methods of treating asthma, allergies, and other pulmonary conditions. TNFalpha, IL-1alpha, IL-1beta, and OX40, have been implicated as contributing to the pathology of asthma, allergies, airway hyperreactivity, and COPD. For example, OX40L deficient mice, or mice treated with anti-OX40L antibody, resist pathological responses to model allergens. IL-1 deficient mice also resist efforts to induce airway hypersensitivity response. TNFalpha is elevated in patients with bronchial hyperreactivity and COPD (see, e.g., Nakae, et al. (2003) Int. Immunol. 15:483-490; Halasz, et al. (2002) Respir. Med. 96:262-267; Chung (2001) Eur. Respir. J. Suppl 34:50s-59s; Hoshino, et al. (2003) Eur. J. Immunol. 333:861-869).

The present invention provides methods to treat cancer by administering an agonist or antagonist to IL-27. Treatment with IL-27 has been found to stimulate expression of cytokines or other signaling molecules associated with anti-tumor response, e.g., TNFalpha, IL-1alpha, IL-1beta, and OX40. Tumor samples expressing increased levels of p28, EBI3, or WSX-1/TCCR indicate that proper immune response to the tumor involves IL-27-mediated signaling, and indicates that the naturally occurring anti-tumor response can be enhanced by administering an agonist of IL-27. Tumor samples expressing increased p28, EBI3, or WSX-1/TCCR, include breast cancer, melanoma, and colon cancer (Table 1).

Other genes in Table 2 have been described. APRIL (A PRoliferation Inducing Ligand) and BLyS are members of the tumor necrosis factor (TNF) ligand family. Lymphotoxin-alpha and beta (LTalpha; LTbeta) are cytokines used in lymph node development (see, e.g., Varfolomeev, et al. (2004) Mol. Cellular Biol. 24:997-1006; Novak, et al. (2002) Blood 100:2973-2979; Nardelli, et al. (2002) Leuk. Lymphoma 43:1367-1373; Shakhov, et al. (2004) Eur. J. Immunol. 34:494-503; Kather, et al. (2003) Immunology 108:338-345).

III. IL-27 Mediates Signaling through WSX-1/TCCR and gp130 .

Various cytokine receptor proteins were paired with WSX-1/TCCR. Only the combination of WSX-1/TCCR with gp130 supported signal transduction in response to IL-27. Neither receptor subunit alone is sufficient to support signal transduction. An anti-human gp130 antibody (anti-hgp130 antibody) blocked IL-27-mediated signaling in a human NK cell line, and IL-27-mediated proliferation of naïve CD4⁺ T cells.

Candidate partner subunits for the WSX-1/TCCR subunit were expressed in mouse pre-B Ba/F3 cells, and assessed for phosphorylation of STAT1 and STAT3. The parental Ba/F3 cell line expresses WSX-1/TCCR (expression relative to ubiquitin was about 100,000) but expresses relatively little gp130 (expression relative to ubiquitin was about 3). Parental Ba/F3 cells and Ba/F3 cells transfected with gp130 were stimulated with IL-3, IL-6/sIL6Ralpha, or IL-27, and assessed for STAT1 phosphorylation. STAT1 was phosphorylated in response to IL-27 only with transfection with gp130 (Table 4). The response of STAT3 to the various stimulants was similar to that of STAT1 (not shown). Thus, IL-27 mediated cell signaling is supported by gp130 in cells naturally expressing WSX-1/TCCR (Table 4).

TABLE 4
Phosphorylation of STAT1 in Ba/F3 cells transfected or not
transfected with gp130. ND means not detectable.
STAT1-P was determined with a specific antibody.

| | Stimulant | | | |
|---|---|---|---|---|
| | | | IL-6/ | |
| | Control | IL-3 | sIL-6Ralpha | IL-27 |
| Type of cell | Phosphorylation of STAT1 | | | |
| Parental Ba/F3 cells, | ND | + | ND | ND |
| transfected with control | | | | |
| plasmid. | | | | |
| Ba/F3 cells transfected | ND | + | + | +++ |
| with plasmid expressing | | | | |
| gp130. | | | | |

Mouse fibroblast cell line NIH3T3 express gp130, where expression of gp130 was much greater than expression of WSX-1/TCCR, i.e., about 1000-fold greater as determined by quantitative PCR analysis (Table 5). The NIH3T3 cells were transfected with a retroviral vector encoding flag-tagged mouse WSX-1/TCCR (mWSX-1/TCCR), a control vector, or not transfected at all. Only cells transfected with WSX-1/TCCR responded to IL-27 by phosphorylation of STAT1 (Table 5). STAT3 phosphorylation was also monitored, and the response results paralleled those of STAT1, except that STAT3 phosphorylation with IL-6/sIL-6Ralpha treatment was somewhat greater than STAT3 phosphorylation with IL-27 treatment. Thus, IL-27-mediated signaling is supported by WSX-1/TCCR in cells naturally expressing gp130 (Table 5).

TABLE 5
Phosphorylation of STAT1 in NIH3T3 cells transfected or not
transfected with flag-tagged mWSX-1. ND means not detectable.
STAT1-P was determined with a specific antibody.

| | Stimulant | | | |
|---|---|---|---|---|
| | | | IL-6/ | |
| | Control | IL-2 | sIL-6Ralpha | IL-27 |
| Type of cell | Phosphorylation of STAT1 | | | |
| Parental NIH3T3 cells, | ND | ND | + | ND |
| transfected with control | | | | |
| plasmid. | | | | |
| NIH3T3 cells transfected | ND | ND | + | +++ |
| with plasmid expressing | | | | |
| WSX-1/TCCR. | | | | |

An anti-human gp130 antibody (anti-hgp130 antibody) was found to block short term and long term response to IL-27, again demonstrating that IL-27 signals through gp130 (Table 6). Short term response was determined with human leukemic natural killer cells (NKL cells), a cell line that responds to IL-27 by tyrosine phosphorylation of STAT1 and STAT3 (Hibbert, et al. (2003) J. Interferon Cytokine Res. 23:513-522). The cells were incubated with and without anti-hgp130 antibody (antibody B-T2) followed by treatment with IL-27 (Wijdenes, et al. (1995) Eur. J. Immunol. 25:3474-3481). NKL cells were preincubated with anti-hgp130 antibody or an isotype control monoclonal antibody. Antibodies were used at 25, 500, and 10,000 ng/ml (Table 6). Cells were stimulated with saturating amounts of IL-27, or left unstimulated. Response to IL-27, and inhibition by the anti-gp130, demonstrates that IL-27 signaling is mediated gp130, were the gp130-mediated signaling provokes phosphorylation of STAT1 and STAT2 (Table 6).

Separate short term studies demonstrated that IL-27 stimulates primary human monocytes to phosphosphorylate STAT1and STAT3 (data not shown), while a time course study, involving time points at t=2h, 6h, and 24h, demonstrated that IL-27 provokes measurable increases in expression of IL-1beta, TNFalpha, and IL-18, only at t=24h (data not shown). Monocytes produce IL-27 in response to IL-27, and express both subunits of the IL-27 receptor, suggesting that monocytes use an autocrine pathway for self-stimulation.

TABLE 6
Anti-gp130 antibody prevents IL-27 mediated cell signaling by
NKL cells. ND means phosphorylation of STAT was not
detectable. Stimulation with IL-27 was for 10-20 min.

| Concentration of added | | | Concentration of added | | |
|---|---|---|---|---|---|
| anti-gp130 antibody | | | isotype control antibody | | |
| 10,000 | | | 10,000 | | |
| ng/ml | 500 ng/ml | 25 ng/ml | ng/ml | 500 ng/ml | 25 ng/ml |

Phosphorylation of STAT1

| | | | | | |
|---|---|---|---|---|---|
| ND | + | ++ | +++ | +++ | +++ |

Phosphorylation of STAT3

| | | | | | |
|---|---|---|---|---|---|
| ND | + | ++ | +++ | +++ | +++ |

Long term effects of IL-27, and the dependence on gp130 for these long term effects, was determined by proliferation assays of naïve human T cells (Table 7). The T cells were purified by by FACS before use in the assays. Proliferation was measured by thymidine incorporation. T cells received IL-27 (saturating levels), agonistic anti-CD3 antibody, agonistic anti-CD28 antibody, and neutralizing anti-IL-2 antibody, as indicated. Cells were titrated with anti-GP130 antibody or with control antibody. [³ H]Thymidine incorporation was a measure of cell proliferation. Maximal incorporation of tritiated thymidine was about 21,000 cpm. Halfmaximal inhibition was found at an anti-gp130 antibody concentration of about 1.0 ng/ml, while maximal inhibition (7,000 cpm) was found at about 30 ng/ml anti-gp130 antibody (Table 7). Where cells were supplemented with medium only, tritium incorporation was zero, i.e., not detectable.

TABLE 7
IL-27-dependent T cell proliferation. (—) means additive not added.

| Additive | | | | | |
|---|---|---|---|---|---|
| | anti-gp 130 | | | | |
| | Ab (about | anti-CD3 | anti-CD28 | anti-IL-2 | [³H]Thymidine |
| IL-27 | 30 ng/ml) | Ab | Ab | Ab | incorporation |
| — | — | — | — | — | zero cpm |
| — | — | yes | yes | yes | 1,500 |
| — | — | yes | yes | — | 6,500 |
| yes | yes | yes | yes | yes | 7,000 |
| yes | — | yes | yes | yes | 21,000 |

IV. Materials and Methods.

Recombinant hIL-6/shIL-6Ralpha, hIL2, and mIL-3 were from R & D Systems, Inc. (Minneapolis, Minn.). Recombinant human and mouse IL-27 fusion proteins are available (Pflanz, et al., supra). Anti-hgp130 monoclonal antibody B-T2 was from the Institute of Biochemistry, RWTH Aachen, Germany. The anti-hWSX-1polyclonal antibody was from U.S. Biological, Swampscott, Mass. Antibodies to tyrosine phosphorylated forms of STAT1 and STAT3 were from Cell Signaling, Beverly, Mass., while antibodies for detecting total STATI or STAT3 were from Transduction Labs, Lexington, Ky., and Santa Cruz Biologicals, Santa Cruz, Calif. Mouse myeloid precursor Ba/F3 cells and human leukemic NK cell line (NKL) were cultured in RPMI 10% fetal calf serum (FCS) in the presence of mIL-3 (5 ng/ml) or hIL-2 (5 ng/ml), respectively. The mouse fibroblast cell line NIH3T3 was cultured in DMEM/10% FCS. Naïve human primary CD4⁺ T cells were prepared and cultured, as described (Pflanz, et al., supra). Freshly isolated human cord blood was separated into mononuclear leukocytes by Ficoll®/Hypaque® centrifugation. Cord blood mononuclear cells were cultured in Yseel's Media (Gemini Bioproducts, Woodland, Calif.) supplemented with 2% human serum, 100 ng/ml stem cell factor, and 50 ng/ml IL-6. Cultures were maintained for about 7-8 weeks with weekly media exchange. At eight weeks, cultures were supplemented with 1 ng/ml of IL-4 and 10 micrograms/ml of human IgE. At 9-10 weeks, the cultures were harvested and residual myeloid cells were removed by magnetic bead depletion of CD15, CD14, and CD11 positive cells (Miltenyi Biotec, Inc., Auburn, Calif.). Mast cell purity (CD117⁺, FcepsilonRI⁺) was verified by FACS analysis to be greater than 97%. Primary human monocytes were obtained by Percoll® density gradient centrifugation from human buffy coat.

STAT tyrosine phosphorylation assays were as follows. Generally, cells were starved 12h in DMEM/2%FCS, then spun down and resuspended to a density of 2.5×10⁶ cells/ml. Cells were stimulated with the respective cytokines at saturating concentrations (100 ng/ml) for 15 min at 37° C., then chilled on ice for 5 min, spun down and resuspended in lysis buffer (2×PBS supplemented with 2mM EDTA, 0.875% Brij 97 (Sigma, St. Louis, Mo.), 0.125% NP40 (Sigma), 1 mM sodium vanadate, 1 mmM sodium fluoride, protease-inhibitor cocktails complete (Roche Applied Science, Indianapolis, Ind.) and 3 mM Pefabloc® (Roche Applied Science). Lysates were centrifuged and supernatants were analyses by SDS-PAGE and subsequent western blot using antibodies described above. NKL cells were incubated with the respective antibody for 20 minutes prior to stimulation with IL-27.

Retroviral infections were as follows. Infection of Ba/F3 and NIH3T3 cells with retroviral constructs encoding the respectively introduced receptors was performed as described (Kitamura (1998) Int. J. Hematol. 67:351-359). Briefly, DNA encoding the mature portion of WSX-1 and the full open reading frame of gp130 was amplified from cDNA libraries (Clontech, Mountain View, Calif.) by standard PCR technology. The gp130 amplicon was cloned into the retroviral vector pMX, the WSX-1 amplicon was cloned 3-prime of a CD8 leader peptide sequence and a flag-tag sequence into pMX vector. Transfection efficiencies with these constructs usually were greater than 80%.

Proliferation assays on naïve CD4⁺ T cells were as follows. FACS sorted CD3⁺CD45RA cells were obtained and subjected to a proliferation experiment with saturating amounts of IL-27 as described (Pflanz, et al., supra). Antibodies were titrated into the assay.

cDNA libraries were analyzed for mRNA expression using a Sybr green protocol (Halfon, et al. (1998) J. Biol. Chem. 273:16400-16408; Bolin, et al. (1997) J. Neurosci. 17:5493-5502). mRNA from Ba/F3 or NIH3T3 cells was prepared using the RNAeasy® kit (Qiagen, Valencia, Calif.). The following forward and reverse PCR primers were used. The primers for human gp130 were from bases 2174-2194 (forward) and bases 2276-2295 (reverse) of GenBank E06613. The primers for mouse gp130 were from bases 1943-1965 (forward) and 2065-2085 (reverse) of GenBank X62646. The primers for mouse WSX-1/TCCR were from bases 1054-1074 (forward) and 1101-1121 (reverse) of GenBank NM_{—}016671. The primers for human WSX/-/TCCR were from bases 1665-1684 (forward primer) and from bases 1726-1746 (reverse primer) of GenBank BC028003.

All citations herein are incorporated herein by reference to the same extent as if each individual publication, patent application, or patent was specifically and individually indicated to be incorporated by reference including all figures and drawings.

Many modifications and variations of this invention, as will be apparent to one of ordinary skill in the art can be made to adapt to a particular situation, material, composition of matter, process, process step or steps, to preserve the objective, spirit and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

1. A method of modulating an immune disorder or condition, comprising administering an effective amount of an agonist or antagonist of p28, EBI3, or WSX/TCCR, wherein the disorder or condition comprises:
a) an inflammatory condition of the skin;
b) arthritis;
c) Crohn's disease;
d) airway hyperreactivity or inflammation;
e) atherosclerosis; or
f) a cancer or tumor not caused by Epstein-Barr virus.

2. The method of claim 1, wherein the antagonist inhibits or prevents binding of IL-27 to a heterodimeric receptor comprising a complex of WSX-1/TCCR and gp130.

3. The method of claim 1, wherein the inflammatory condition of the skin comprises:
a) psoriasis; or
b) atopic dermatitis.

4. The method of claim 1, wherein the arthritis comprises:
a) rheumatoid arthritis;
b) osteoarthritis; or
c) psoriatic arthritis.

5. The method of claim 1, wherein the airway hyperreactivity or inflammation disorder comprises:
a) asthma;
b) allergy; or
c) chronic obstructive pulmonary disorder (COPD).

6. The method of claim 1, wherein the cancer or tumor comprises:
a) breast cancer;
b) colon cancer; or
c) melanoma.

7. The method of claim 1, wherein the agonist inhibits or ameliorates the disorder comprising a cancer or tumor.

8. The method of claim 6, wherein the cancer or tumor expresses detectably increased amounts, relative to expression by a normal, control tissue, of:
a) p28;
b) EBI3; or
c) or WSX-1/TCCR.

9. The method of claim 1, wherein the antagonist ameliorates the:
a) inflammatory condition of the skin;
b) arthritis;
c) Crohn's disease;
d) airway hyperreactivity or airway inflammation; or
e) atherosclerosis.

10. The method of claim 1, wherein the agonist comprises:
a) IL-27;
b) IL-27 hyperkine;
c) p28;
d) EBI3; or
e) a nucleic acid.

11. The method of claim 10, wherein the nucleic acid encodes:
a) IL-27 hyperkine;
b) p28;
c) EBI3;
d) a first p28 polypeptide chain and a second EBI3 polypeptide chain;
e) WSX-1/TCCR; or
f) WSX/1/TCCR and gp130 .

12. The method of claim 1, wherein the antagonist comprises a binding composition from an antibody that specifically binds:
a) IL-27;
b) p28;
c) EBI3;
d) WSX-1/TCCR; or
e) a complex of gp130 and WSX-1/TCCR.

13. The method of claim 12, wherein the binding composition from an antibody comprises:
a) a polyclonal antibody;
b) a monoclonal antibody;
c) a humanized antibody, or a fragment thereof;
d) an Fab, Fv, or F(ab′)₂ fragment;
e) a peptide mimetic of an antibody; or
f) a detectable label.

14. The method of claim 1, wherein the antagonist comprises:
a) a soluble receptor derived from WSX-1/TCCR;
b) a small molecule; or
c) a nucleic acid.

15. The method of claim 14, wherein the nucleic acid specifically hybridizes with a polynucleotide encoding:
a) p28;
b) EBI3; or
c) WSX-1/TCCR.

16. The method of claim 15, wherein the nucleic acid comprises:
a) anti-sense nucleic acid; or
b) small interference RNA (siRNA).

17. The method of claim 1, wherein administration of the agonist increases expression of:
a) RANKL;
b) TNFalpha;
c) TEASRL;
d) IL-1alpha or beta;
e) OX40; or
f) APRIL.

18. The method of claim 1, wherein administration of the antagonist decreases expression of:
a) RANKL;
b) TNFalpha;
c) TEASRL;
d) IL-1alpha or beta;
e) OX40; or
f) APRIL.

19. A method of diagnosing the immune condition or disorder of claim 1, comprising contacting a binding composition to a biological sample, wherein the binding composition specifically binds to:
a) IL-27, p28, EBI3, or WSX-1/TCCR;
b) a complex of WSX-1/TCCR and gp130; or
c) a nucleic acid encoding p28, EBI3, or WSX-1/TCCR;
and measuring or determining the specific binding of the binding composition to the biological sample.

20. A kit for the diagnosis of the immune condition or disorder of claim 1, comprising a compartment and a binding composition that specifically binds to:
a) IL-27, p28, EBI3, or WSX-1/TCCR;
b) a complex of WSX-1/TCCR and gp130; or
c) a nucleic acid encoding p28, EBI3, or WSX-1/TCCR;
